SEQUENCE LISTING (11 sequences)

SEQ ID NO: 1 - DNA, 1618 bp, Mus musculus
  ref <308>: DATABASE ACCESSION NUMBER: AF480491.1
  ref <309>: DATABASE ENTRY DATE: 2002-03-05
  ref <313>: RELEVANT RESIDUES IN SEQ ID NO: (1)..(1618)
tcaacaagaaggagctgtgggacaggagctaatacccagaactgagttgtgtcctgctaagtcctctgccacgtacccacgggatgaagaacctttcatttcccctccttttccttttcttccttgtccctgaactgctgggctccagcatgccactgtgtcccatcgatgaagccatcgacaagaagatcaaacaagacttcaactccctgtgtgaggatccccccttcgcccccatccaaccttgccgagggccttacagacttatccctgatggggaacagacccgcccagctacaaggctaccctcagcctcagcctcaccctgttccaacccaaattcatcctgacctaccttgatcatggttcaccccacaacttagcttcttccaaccatcacctccaaacctcccctcactccaaaaggccccacactgtggtccatggagctgagcccccagtgcctgaccctcttgcctcagcctcctgagtgtttggagtacaggtgtgagcaaaatattttaccgtgaacgtgtgctgtccatgtctgtgtatgtgtgtttatatatgtggtgcacatatgtgaaggagcttgtgtgtgcataggcatatggagaattgaagttggtgtagtgttatcattccacctctgctttgttgagacagaatctctcagctgaacccagggcttgtgggtttgtggtagttctagttagccatcttgttctggggactccatgtctgtctgcctcctggaggctgggattatgggtgcccctacaccatgcatgcctagcttctaaatgggttctggggatcttggctctgatcctcccacttgtgagtcaaactctttagctataagtcaccttctcactctgctgagaagaatttgacctcactgtcagtgtcaaacccaaagatcgccccctgcaaagatgcagctggattaccccttcaatcttcccttctgcagttccaaatgcaataaagaacattggcttaaattgctggacagtctcctccagagggaagttggcctcctgcccagaaggtgagtgcaaactctgttgttcagactttctggaagttctgaaactcctctgtctgccttgtctgggtcctcaacatgtctgtccttaggactccctcccagacttccagtcttctcatctcccagcccagctcaaggtcctttagactcctgctgtatcatcttgcactctgtttccgtggcaacagcctccagccccctcccccatctctgcctcccacctgccacagtcttccggtttttcagggacttctttccttgcaggcacagcagtcttgagctgctcctgtggctctgcctgtggctcgtgggacattcgtgaagaaaaagtgtgtcactgccagtgtgcaaggatagactggacagcagcccgctgctgtaagctgcaggtcgcttcctgatgtcggggaagtgagcgtggtttccagcacagccacccgttcctgtagctccagagatgtctgatgtcctccggtctctacaggcacctgcactcacgtgcgcgaatccacacacaagcacacatacttaaaaataaaacaaaacaggctggaaaaaaaaaaa

SEQ ID NO: 2 - DNA, 607 bp, Mus musculus
  ref <308>: DATABASE ACCESSION NUMBER: NM_001204959.1
  ref <309>: DATABASE ENTRY DATE: 2013-04-14
  ref <313>: RELEVANT RESIDUES IN SEQ ID NO: (1)..(607)
gtcaacaagaaggagctgtgggacaggagctaatacccagaactgagttgtgtcctgctaagtcctctgccacgtacccacgggatgaagaacctttcatttcccctccttttccttttcttccttgtccctgaactgctgggctccagcatgccactgtgtcccatcgatgaagccatcgacaagaagatcaaacaagacttcaactccctgtttccaaatgcaataaagaacattggcttaaattgctggacagtctcctccagagggaagttggcctcctgcccagaaggcacagcagtcttgagctgctcctgtggctctgcctgtggctcgtgggacattcgtgaagaaaaagtgtgtcactgccagtgtgcaaggatagactggacagcagcccgctgctgtaagctgcaggtcgcttcctgatgtcggggaagtgagcgtggtttccagcacagccacccgttcctgtagctccagagatgtctgatgtcctccggtctctacaggcacctgcactcacgtgcgcgaatccacacacaagcacacatacttaaaaataaaacaaaacaggctggaaaaaaaaaaaaaaaaaaaaaaaaaa

SEQ ID NO: 3 - protein, 114 aa, Mus musculus
  ref <308>: DATABASE ACCESSION NUMBER: AAG59823.1
  ref <309>: DATABASE ENTRY DATE: 2001-01-28
  ref <313>: RELEVANT RESIDUES IN SEQ ID NO: (1)..(114)
MKNLSFPLLFLFFLVPELLGSSMPLCPIDEAIDKKIKQDFNSLFPNAIKNIGLNCWTVSSRGKLASCPEGTAVLSCSCGSACGSWDIREEKVCHCQCARIDWTAARCCKLQVAS

SEQ ID NO: 4 - protein, 21 aa, Homo sapiens
  ref <308>: DATABASE ACCESSION NUMBER: 1T7H_B
  ref <309>: DATABASE ENTRY DATE: 2012-10-10
  ref <313>: RELEVANT RESIDUES IN SEQ ID NO: (3)..(18)
  ref <308>: DATABASE ACCESSION NUMBER: 1T7H_A
  ref <309>: DATABASE ENTRY DATE: 2012-10-10
  ref <313>: RELEVANT RESIDUES IN SEQ ID NO: (3)..(18)
  ref <308>: DATABASE ACCESSION NUMBER: 1EDP_A
  ref <309>: DATABASE ENTRY DATE: 2012-10-10
  ref <313>: RELEVANT RESIDUES IN SEQ ID NO: (1)..(17)
CSCSSLMDKECVYFCHLDIIW

SEQ ID NO: 5 - protein, 212 aa, Homo sapiens
  ref <308>: DATABASE ACCESSION NUMBER: P05305.1
  ref <309>: DATABASE ENTRY DATE: 2013-07-24
  ref <313>: RELEVANT RESIDUES IN SEQ ID NO: (1)..(212)
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 6 - protein, 380 aa, Mus musculus
  ref <308>: DATABASE ACCESSION NUMBER: Q63844.5
  ref <309>: DATABASE ENTRY DATE: 2013-07-24
  ref <313>: RELEVANT RESIDUES IN SEQ ID NO: (1)..(380)
MAAAAAAPGGGGGEPRGTAGVVPVVPGEVEVVKGQPFDVGPRYTQLQYIGEGAYGMVSSAYDHVRKTRVAIKKISPFEHQTYCQRTLREIQILLRFRHENVIGIRDILRAPTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFDMELDDLPKERLKELIFQETARFQPGAPEGP

SEQ ID NO: 7 - protein, 358 aa, Mus musculus
  ref <308>: DATABASE ACCESSION NUMBER: NP_001033752.1
  ref <309>: DATABASE ENTRY DATE: 2013-08-19
  ref <313>: RELEVANT RESIDUES IN SEQ ID NO: (1)..(358)
MAAAAAAGPEMVRGQVFDVGPRYTNLSYIGEGAYGMVCSAYDNLNKVRVAIKKISPFEHQTYCQRTLREIKILLRFRHENIIGINDIIRAPTIEQMKDVYIVQDLMETDLYKLLKTQHLSNDHICYFLYQILRGLKYIHSANVLHRDLKPSNLLLNTTCDLKICDFGLARVADPDHDHTGFLTEYVATRWYRAPEIMLNSKGYTKSIDIWSVGCILAEMLSNRPIFPGKHYLDQLNHILGILGSPSQEDLNCIINLKARNYLLSLPHKNKVPWNRLFPNADSKALDLLDKMLTFNPHKRIEVEQALAHPYLEQYYDPSDEPIAEAPFKFDMELDDLPKEKLKELIFEETARFQPGYRS

SEQ ID NO: 8 - protein, 447 aa, Mus musculus
  ref <308>: DATABASE ACCESSION NUMBER: CAC88132.1
  ref <309>: DATABASE ENTRY DATE: 2006-11-14
  ref <313>: RELEVANT RESIDUES IN SEQ ID NO: (1)..(447)
MSDSKSDGQFYSVQVADSTFTVLKRYQQLKPIGSGAQGIVCAAFDTVLGINVAVKKLSRPFQNQTHAKRAYRELVLLKCVNHKNIISLLNVFTPQKTLEEFQDVYLVMELMDANLCQVIHMELDHERMSYLLYQMLCGIKHLHSAGIIHRDLKPSNIVVKSDCTLKILDFGLARTACTNFMMTPYVVTRYYRAPEVILGMGYKENVDIWSVGCIMAEMVLHKCLFPGRDFDIWSVGCIMGELVKGCVIFQGTDHIDQWNKAIEQLGTPSAEFMKKLQPTVRNYVENRPKYPGIKFEELFPDWIFPSESERDKIKTSQARDLLSKMLVIDPDKRISVDEALRHPYITVWYDPAEAEAPPPQIYDAQLEEREHAIEEWKELIYKEVMDWEERSKNGVKDQPSDAAVSSKATPSQSSSINDISSMSTEHTLASDTDSSLDASTGPLEGCR

SEQ ID NO: 9 - protein, 480 aa, Mus musculus
  ref <308>: DATABASE ACCESSION NUMBER: P31750.2
  ref <309>: DATABASE ENTRY DATE: 2013-07-24
  ref <313>: RELEVANT RESIDUES IN SEQ ID NO: (1)..(480)
MNDVAIVKEGWLHKRGEYIKTWRPRYFLLKNDGTFIGYKERPQDVDQRESPLNNFSVAQCQLMKTERPRPNTFIIRCLQWTTVIERTFHVETPEEREEWATAIQTVADGLKRQEEETMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPTQRLGGGSEDAKEIMQHRFFANIVWQDVYEKKLSPPFKPQVTSETDTRYFDEEFTAQMITITPPDQDDSMECVDSERRPHFPQFSYSASGTA

SEQ ID NO: 10 - protein, 770 aa, Mus musculus
  ref <308>: DATABASE ACCESSION NUMBER: AAA19452.1
  ref <309>: DATABASE ENTRY DATE: 1994-07-01
  ref <313>: RELEVANT RESIDUES IN SEQ ID NO: (1)..(770)
MAQWNQLQQLDTRYLKQLHQLYSDTFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMDLTSECATSPM

SEQ ID NO: 11 - protein, 427 aa, Mus musculus
  ref <308>: DATABASE ACCESSION NUMBER: Q61614.3
  ref <309>: DATABASE ENTRY DATE: 2013-07-24
  ref <313>: RELEVANT RESIDUES IN SEQ ID NO: (1)..(427)
MSIFCLAAYFWLTMVGGVMADNPERYSANLSSHMEDFTPFPGTEINFLGTTHRPPNLALPSNGSMHGYCPQQTKITTAFKYINTVISCTIFIVGMVGNATLLRIIYQNKCMRNGPNALIASLALGDLIYVVIDLPINVFKLLAGRWPFDHNDFGVFLCKLFPFLQKSSVGITVLNLCALSVDRYRAVASWSRVQGIGIPLITAIEIVSIWILSFILAIPEAIGFVMVPFEYKGELHRTCMLNATSKFMEFYQDVKDWWLFGFYFCMPLVCTAIFYTLMTCEMLNRRNGSLRIALSEHLKQRREVAKTVFCLVVIFALCWFPLHLSRILKKTVYDEMDKNRCELLSFLLLMDYIGINLATMNSCINPIALYFVSKKFKNCFQSCLCCCCHQSKSLMTSVPMNGTSIQWKNQEQNNHNTERSSHKDSMN

(19) United States
(12) Patent Application Publication
(10) Pub. No.: US 2015/0045358 A1
(43) Pub. Date: Feb. 12, 2015

(54) PHARMACEUTICAL COMPOSITION FOR TREATING DISORDERS ASSOCIATED WITH INSULIN RESISTANCE

(71) Applicant: National Central University, Jhongli City (TW)

(72) Inventors: Yung-hsi KAO, Jhongli City (TW); Ya-chu Tang, Jhongli City (TW); Hsin-huei Chang, Jhongli City (TW); Hui-chen Ku, Jhongli City (TW)

(21) Appl. No.: 13/898,524

(22) Filed: May 21, 2013

(30) Foreign Application Priority Data
Nov. 21, 2012 (TW) 101143523

(51) Int. Cl. A61K 31/5377; A61K 31/366; A61K 31/416; A61K 31/277; A61K 31/352

(52) U.S. Cl. CPC A61K 31/5377; A61K 31/277; A61K 31/352; A61K 31/416; A61K 31/366
USPC 514/233.5; 558/396; 549/403; 548/356.5; 544/151; 549/275; 558/392; 514/523; 514/456; 514/403; 514/453; 514/521

(57) ABSTRACT

A pharmaceutical composition for treating disorders associated with insulin resistance is disclosed, and the composition comprises at least one inhibitor which is an effective agent to suppress endothelin-1-stimulated resistin gene expression through decreasing the endothelin-1-stimulated phosphorylation of proteins downstream of endothelin type A receptor, wherein the downstream signaling molecules comprise ERK1/2, JNKs, AKT, and STAT3 proteins, and wherein the inhibitor is selected from at least one antagonist of the endothelin type A receptor or downstream signaling proteins.

### CROSS-REFERENCE

This application claims the priority of Taiwan Patent Application No. 101143523, filed on Nov. 21, 2012. This invention is partly disclosed in a thesis entitled “Endothelin-1 up-regulates resistin gene expression in 3T3-L1 adipocytes” on May 21, 2012 completed by Ya-Chu Tang.

### FIELD OF THE INVENTION

The present invention relates to a pharmaceutical composition for treating disorders associated with insulin resistance in a particular way to decrease the endothelin(ET)-1 -stimulated phosphorylation of downstream signaling molecules.

### BACKGROUND OF THE INVENTION

Insulin resistance means that insulin sensitization of peripheral tissue or organ is reduced and thereby leading to a pathological condition of the body. insulin resistance is reported to cause other metabolism-relevant diseases, including hypertension, glucose intolerance, dyslipidemia, atherosclerosis, microalbuminuria, hypercoagulability, central obesity and other cardiovascular diseases. Therefore, insulin resistance is also called metabolic syndrome. Clinically, the patient has type II diabetes mellitus; before the disease happens, insulin resistance will occur. If insulin resistance syndrome is not treated or improved in time, it could lead to the incidence of type II diabetes mellitus. That is, the patient with insulin resistance syndrome is the high risk group of type II diabetes mellitus. Therefore, if the patient with insulin resistance can be treated earlier, it will prevent or delay the later development of various metabolic syndromes and will eventually save medical costs and resources.

In recent years, the research of insulin resistance has shown that obesity causes the generation of chronic inflammation. In particular, the pro-inflammatory cytokines secreted from the adipocytes are thought to be an important factor of linking obesity and insulin resistance. In addition, the clinical report has indicated that the patients suffering from hypertension and/or insulin resistance have higher plasma ET-1 level than the normal subjects. ET-1 is known as one of the strongest vasoconstrictors, and it possesses many important physiological functions. For example, ET-1 regulates the cardiovascular function, maintains the basic vascular tension, and stabilizes the cardiovascular system. Accordingly, if a strategy is used for treatment of insulin resistance directly by decreasing the level of ET-1 protein, the result will have a direct effect on the stability of the cardiovascular system.

In order to solve the problems as described above, it is necessary to provide a medical composition to treat insulin resistance related diseases.

### SUMMARY OF THE INVENTION

The objective of the present invention is to provide a pharmaceutical composition for treating disorders associated with insulin resistance. This composition comprises at least one inhibitor which decreases the activity of an endothelin receptor and the phosphorylation of downstream signaling molecules stimulated by ET-1. Without affecting ET-1 protein expression, the inhibitor is able to suppress the ET-1-increased levels of resistin mRNA expression through decreasing the ET-1-stimulated phosphorylation of proteins downstream of endothelin type A receptor (ET_{A}R).

To achieve the above objective, the present invention selects the following ET-1 signaling molecules, such as endothelin receptor, extracellular signal-regulated kinase (ERK1/2), c-Jun amino-terminal kinases (JNKs), protein kinase B (AKT), and signal transducer and activator of transcription 3 (STATS), and at least one inhibitor of them.

In one embodiment of the present invention, a pharmaceutical composition for treating disorders associated with insulin resistance, comprises: at least one inhibitor for decreasing the phosphorylation of downstream signaling molecules stimulated by ET-1, in order to be an effective component to inhibit the gene expression level of resistin, wherein the downstream signaling molecules comprise ERK1/2, JNKs, AKT or STAT3, and wherein the inhibitor is selected from at least one antagonist of the ET-1 signaling molecule.

In one embodiment of the present invention, the inhibitor is selected from an inhibitor of ET_{A}R.

In one embodiment of the present invention, the ERK1/2 antagonist is selected from either 1,4-diamino-2,3-dicyano-1,4-bis (o-aminophenylmercapto) butadiene (U0126) or 2-(2-amino-3-methoxyphenyl)-4H-1-benzopyran-4-one (PD98059).

In one embodiment of the present invention, the JNKs antagonist is selected from anthrapyrazolone (SP600125).

In one embodiment of the present invention, the AKT antagonist is selected from either 2-(4-morpholinyl)-8-phenyl-4H-1-benzopyran-4-one (LY294002) or wortmannin.

In one embodiment of the present invention, the STAT3 antagonist is tyrphostin (AG490).

In one embodiment of the present invention, the disorders associated with insulin resistance is selected from hypertension, inflammation, metabolic syndrome, dyslipidemia, glucose intolerance, type II diabetes, hyperuricemia, central obesity, blood coagulation system defects, high blood coagulation, hyperandrogenism, fatty liver, or coronary heart disease.

In one embodiment of the present invention, the pharmaceutical composition further comprises at least one of pharmaceutically acceptable carriers, diluents, carrier substances, and adjuvants.

In one embodiment of the present invention, the pharmaceutical composition is a formulation of either oral type, intramuscular injection type, or intravenous injection type.

### DESCRIPTION OF THE DRAWINGS

FIG. 1A is a computer image of resistin mRNA level according to a pharmaceutical composition for treating disorders associated with insulin resistance of a preferred embodiment of the present invention;

FIG. 1B is a histogram of resistin mRNA level according to a pharmaceutical composition for treating disorders associated with insulin resistance of the preferred embodiment of the present invention;

FIG. 1C is a computer image of resistin mRNA level according to a pharmaceutical composition for treating disorders associated with insulin resistance of the preferred embodiment of the present invention;

FIG. 1D is a histogram of resistin mRNA level according to a pharmaceutical composition for treating disorders associated with insulin resistance of the preferred embodiment of the present invention;

FIG. 2A is a histogram of ERK1/2 protein phosphorylation level according to a pharmaceutical composition for treating disorders associated with insulin resistance of the preferred embodiment of the present invention;

FIG. 2B is a histogram of JNKs protein phosphorylation level according to a pharmaceutical composition for treating disorders associated with insulin resistance of the preferred embodiment of the present invention;

FIG. 2C is a histogram of p38 protein phosphorylation level according to a pharmaceutical composition for treating disorders associated with insulin resistance of the preferred embodiment of the present invention;

FIG. 2D is a histogram of AKT protein phosphorylation level according to a pharmaceutical composition for treating disorders associated with insulin resistance of the preferred embodiment of the present invention;

FIG. 2E is a histogram of STAT3 protein phosphorylation level according to a pharmaceutical composition for treating disorders associated with insulin resistance of the preferred embodiment of the present invention;

FIG. 3A is a histogram of endothelin receptor inhibitors affecting the phosphorylation level of downstream signaling molecules according to a pharmaceutical composition for treating disorders associated with insulin resistance of the preferred embodiment of the present invention;

FIG. 3B is a histogram of endothelin receptor inhibitors affecting the phosphorylation level of downstream signaling molecules according to a pharmaceutical composition for treating disorders associated with insulin resistance of the preferred embodiment of the present invention;

FIG. 3C is a histogram of endothelin receptor inhibitors affecting resistin mRNA level according to a pharmaceutical composition for treating disorders associated with insulin resistance of the preferred embodiment of the present invention;

FIG. 4A is a histogram of ERK1/2 protein phosphorylation level affected either by U0126 or by PD98059 according to a pharmaceutical composition for treating disorders associated with insulin resistance of the preferred embodiment of the present invention;

FIG. 4B is a histogram of JNKs protein phosphorylation level affected by SP600125 according to a pharmaceutical composition for treating disorders associated with insulin resistance of the preferred embodiment of the present invention;

FIG. 4C is a histogram of p38 protein phosphorylation level affected by SB203580 according to a pharmaceutical composition for treating disorders associated with insulin resistance of the preferred embodiment of the present invention;

FIG. 4D is a histogram of AKT protein phosphorylation level affected either by LY294002 or by wortmannin according to a pharmaceutical composition for treating disorders associated with insulin resistance of the preferred embodiment of the present invention;

FIG. 4E is a histogram of STAT3 protein phosphorylation level affected by AG490 according to a pharmaceutical composition for treating disorders associated with insulin resistance of the preferred embodiment of the present invention; and

FIG. 4F is a histogram of resistin protein level affected by ET-1 according to a pharmaceutical composition for treating disorders associated with insulin resistance of the preferred embodiment of the present invention.

### DETAILED DESCRIPTION OF THE PREFERRED EMBODIMENTS

In order to clearly understand the meaning of each figure as indicated above, as well as the objectives, features and advantages of the present invention, the following specifications will be provided with the preferred embodiment accompanied with the drawings and with the detailed descriptions.

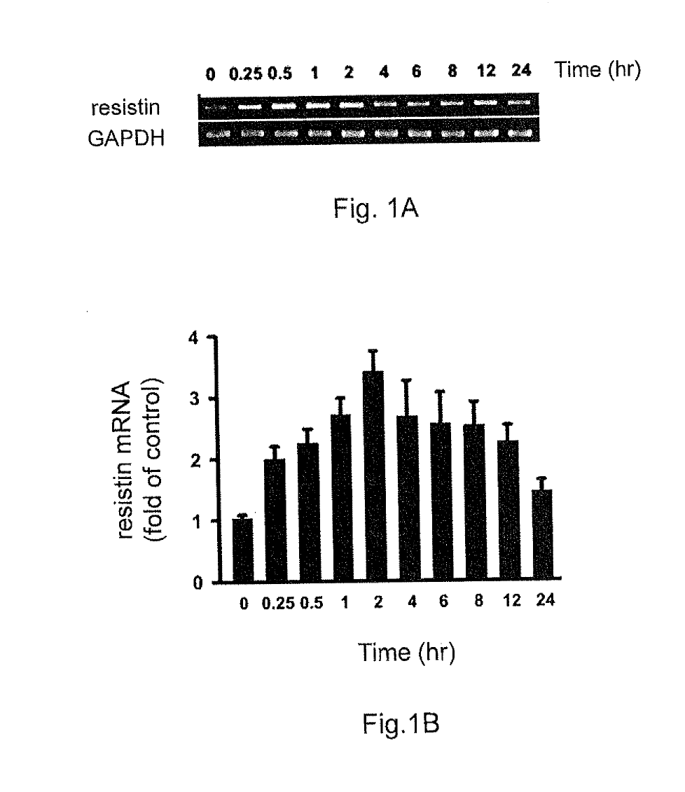

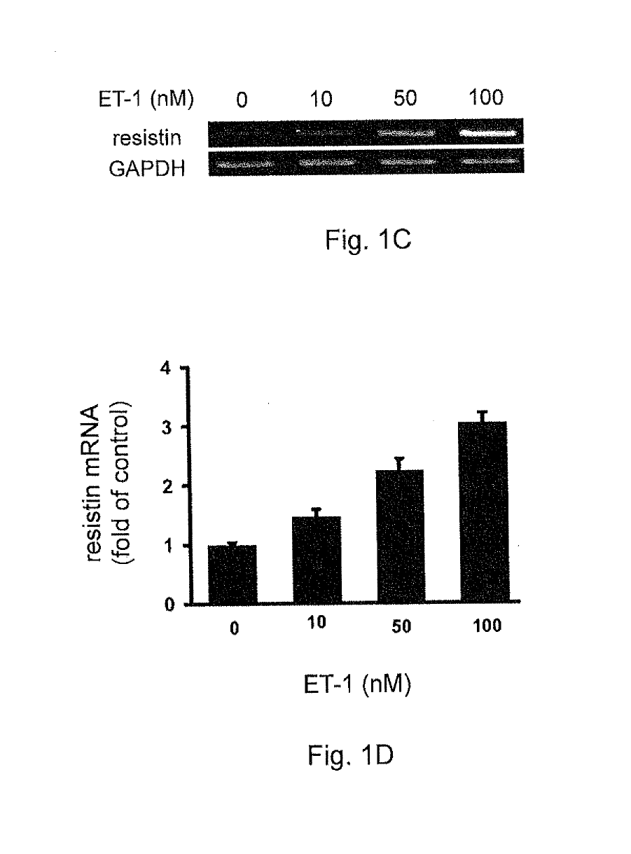

Referring to FIGS. 1A and 1B which are presented to detect the time-dependent effect of ET-1 on resistin mRNA level, the differentiated 3T3-L1 adipocytes are treated with 100 nM ET-1 at different time periods, including 0, 0.25, 0.5, 1, 2, 4, 6, 8, 12, and 24 hours, After the treatment, resistin mRNA is analyzed by the respective method of reverse transcription-polymerase chain reaction (RT-PCR) (FIG. 1A) and real time-PCR. The forward and reverse primers are 5′-CCTCTGGAAAGCTGTGGCGT-3′ and 5′-TTGGCAGGTTTCTCCAGGCG-3′ for mouse GAP DH and 5′-AAGCCATCAACAAGAAGATCAAA-3′ and 5′-TCCAGCAATTTAAGCCAATGTTC-3′ for mouse resistin, respectively. The statistic graph presented in FIG. 1B shows that resistin mRNA levels are increased by 100-250% at 0.25-12 hours after ET-1 treatment. The duration of ET-1 incubation that causes the maximum effect on resistin mRNA levels is 2 hours.

Referring to FIGS. 1C and 1D which are presented to detect the concentration-dependent effect of ET-1 on resistin mRNA level, the differentiated 3T3-L1 adipocytes are treated with different concentrations of ET-1, such as 0, 10, 50, and 100 nM. After 2 hours of ET-1 incubation, resistin mRNA levels are determined by RT-PCR (FIG. 1C) and by real time-PCR (FIG. 1D). Levels of resistin mRNA are increased by 50%, 100%, and 200% after 10, 50 and 100 nM ET-1 are respectively added. According to the results, 100 nM ET-1 is used in the following embodiments of the present invention for treatment of the differentiated 3T3-L1 adipocytes.

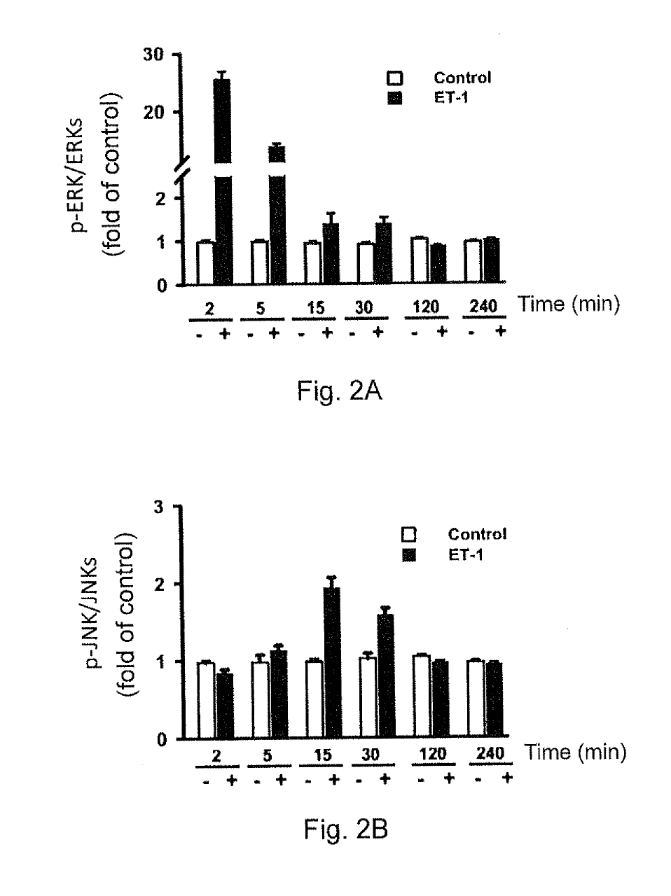

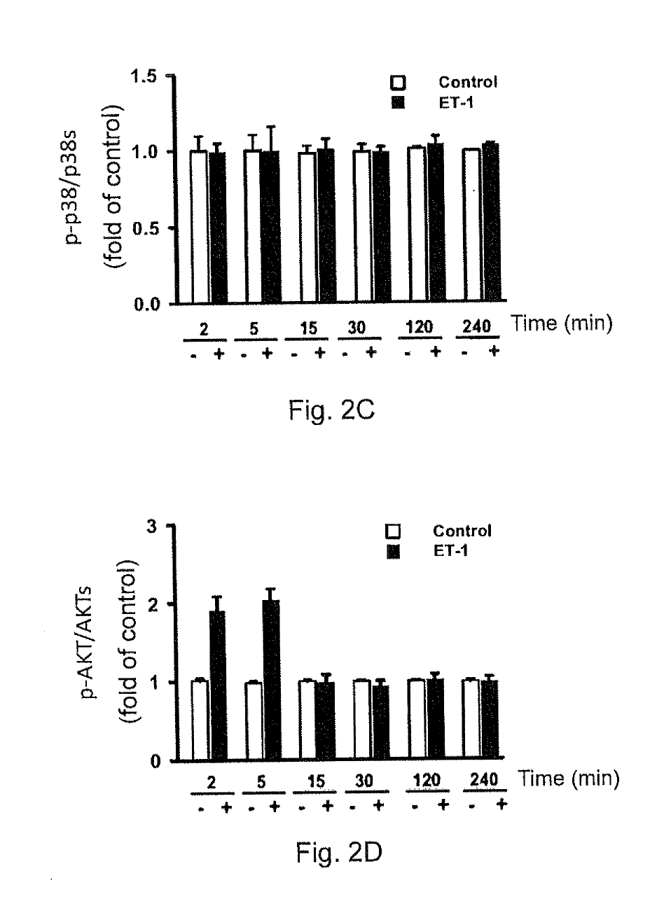

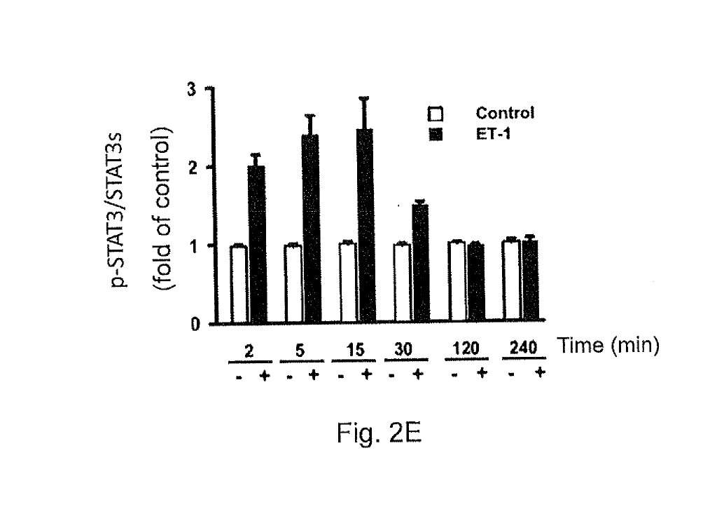

Referring to FIGS. 2A, 2B, 2C, 2D, and 2E which are presented to detect the time-dependent effect of ET-1 on ET-1 signaling molecules, including ERK1 /2, JNKs, p38, MT or STAT3 proteins, the differentiated 3T3-L1 adipocytes are treated with or without 100 nM ET-1 at different time points, such as 2, 5, 15, 30, 120, and 240 minutes. After ET-1 incubation, the total protein amounts and the phosphorylation levels of each ET-1 signaling molecule are determined by Western blotting analysis. ET-1 induced the maximum phosphorylation of the ERK1/2 at 2 minutes (FIG. 2A), the JNKs at 15 minutes (FIG. 26), the AKT at 2 and 5 minutes (FIG. 2D), and the STAT3 at 2, 5 and 15 minutes (FIG. 2E). Additionally, the phosphorylation of the p38 is not altered by ET-1 stimulation. According to the above results, the inhibitors of these ET-1 signaling molecules are used in the present invention to investigate the stimulatory effect of ET-1 on resistin mRNA expression.

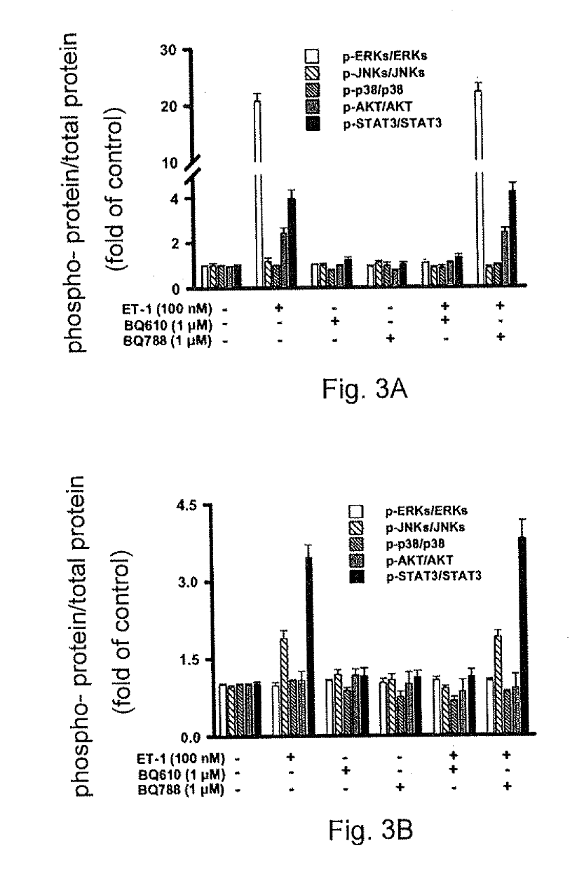

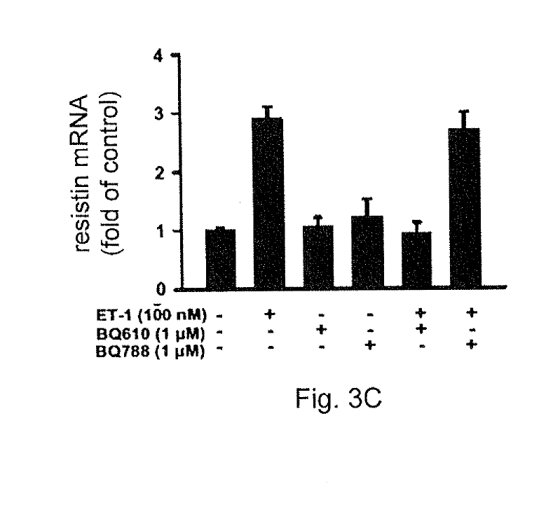

Referring to FIGS. 3A, 3B, and 3C which are presented to further study the endothelin receptor-dependent of ET-1 on the phosphorylation of downstream signaling molecules and on resistin mRNA expression, the differentiated 3T3-L1 adipocytes are pretreated with or without either 1 μM BQ610 (an ET_{A}R inhibitor) or 1 μM BQ788 (an endothelin type B receptor (ET_{B}R) inhibitor) for 1 hour, and then incubated with 100 nM ET-1. After different time points of ET-1 treatment, ET-1 signaling molecules, such as ERK1/2, JNKs, P38, AKT, and STAT3 proteins, and resistin mRNA levels are determined by Western blotting analysis and PCR analysis, respectively. FIG. 3A, 3B, and 3C are presented with or without 5, 15, and 120 minutes of 100 nM ET-1 treatments, respectively.

In FIGS. 3A and 3B, ET-1 alone for 5 minutes or 15 minutes stimulates the activity of endothelin receptor, as indicated by increased phosphorylation of downstream signaling molecules, such as ERK1/2, JNKs, AKT, or STAT-3 proteins. BQ610 at 1 μM alone does not alter the basal level of the phosphorylation of ET-1 signaling molecules; in the presence of ET-1, it inhibits the ET-1-stimulated phosphorylation of ERK1/2, JNKs, AKT, or STAT3. In the presence and absence of ET-1, BQ788 at 1 μM does not alter the total amounts of ERK1/2, JNKs, AKT and STAT3 proteins or the phosphorylation of these ET-1 signaling molecules. Therefore, the increase in the phosphorylation levels of ET-1 signaling molecules induced by ET-1 is mediated through the ET_{A}R-dependent and ET_{B}R-independent pathways. In FIG. 3C, pretreatment with either 1-μM BQ610 or 1-μM BQ788 alone does not alter resistin mRNA level In the presence of ET-1, BQ610, but not BQ788, suppresses the ET-1-stimulated resistin mRNA expression. These observations suggest that ET-1 increases resistin mRNA level through the ET_{A}R but not ET_{B}R pathways.

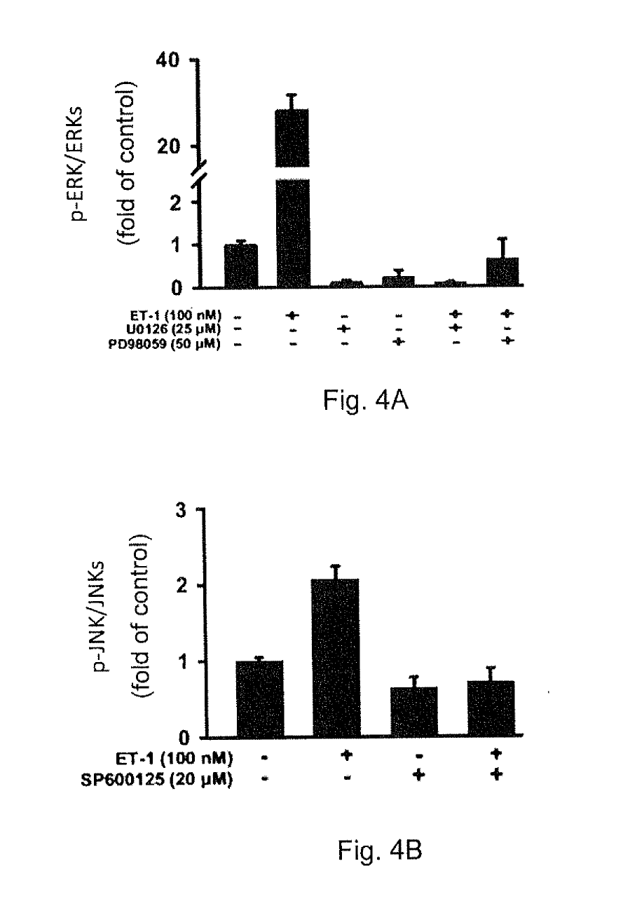

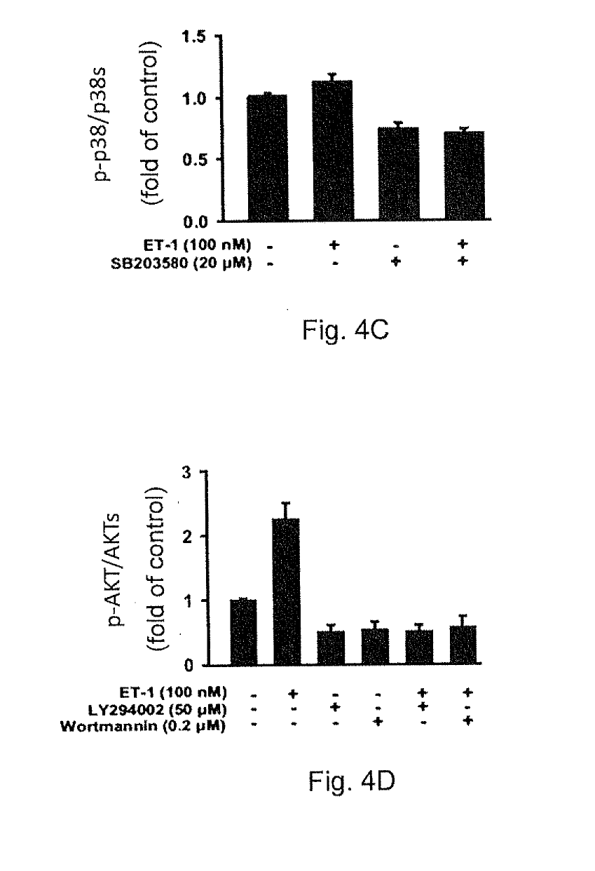

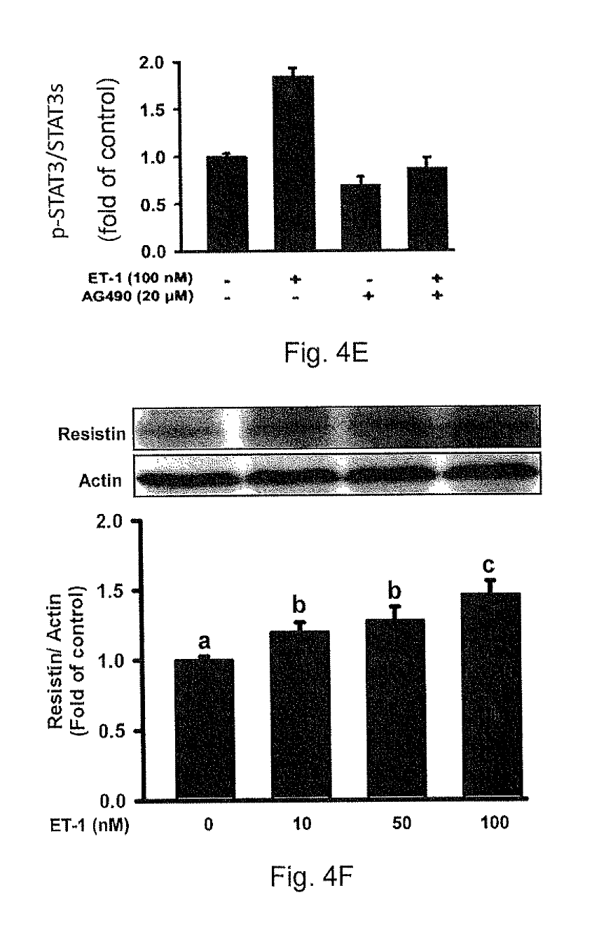

Referring to FIGS. 4A, 4B, 4C, 4D, and 4E which are presented to study the effect of the inhibitors of ET-1 signaling molecules, the differentiated 3T3-L1 adipocytes are pretreated with or without the specific inhibitors of ERK1/2, JNK, AKT, STAT3, or p38 for 30 minutes and then incubated with 100 nM ET-1. These inhibitors comprise 1,4-diamino-2,3-dicyano-1,4-bis [2-aminophenylthio]butadiene (U0126, 25 μM; an ERK1/2 inhibitor), 2-(2-Amino-3-methoxyphenyl)-4H-1-benzopyran-4-one (PD98059, 50 μM; an ERK1/2 inhibitor), anthrapyrazolone (SP600125, 20 μM; a JNK inhibitor), SB203580 (20 μM; a p38 inhibitor), 2-(4-morpholinyl)-8-phenyl-4H-1-benzopyran-4-one (LY294002, 50 μM; a PI3K/AKT inhibitor), wortmannin (0.2 μM; a PI3K/AKT inhibitor), and tyrphostin (AG490, 20 μM; a STAT3 inhibitor). After 5, 15 and 120 minutes of 100-nM ET-1 treatment, the total amounts and the phosphorylation level of the above ET-1 signaling molecules corresponding to the ET-1 stimulated increases in resistin mRNA level are determined by Western blotting analysis. In FIG. 4A, the ET-1-stimulated phosphorylation of the ERK1/2 proteins is inhibited either by 1,4-diamino-2,3-dicyano-1,4-bis[2-aminophenylthio]butadiene (U0126, 25 μM) or by 2-(2-Amino-3-methoxyphenyl)-4H-1-benzopyran-4-one (PD98059, 50 μM). In FIG. 4B, the ET-1-stimulated phosphorylation of JNKs is inhibited by anthrapyrazolone (SP600125, 20 μM). In FIG. 4C, the phosphorylation of p38 is not stimulated by ET-1 or inhibited by SB203580 inhibitor (20 μM). In FIG. 40, the ET-1-stimulated phosphorylation of AKT is inhibited either by 2-(4-morpholinyl)-8-phenyl-4H-1-benzopyran-4-one (LY294002, 50 μM) or by wortmannin (0.2 μM). In FIG. 4E, the ET-1-stimulated phosphorylation of STAT3 is inhibited by tyrphostin (AG490, 20 μM).

Referring to FIG. 4F which detects the concentration-dependent effect of ET-1 on resistin protein level, the differentiated 3T3-L1 adipocytes are treated with different concentrations of ET-1, such as 0, 10, 50, and 100 nM. After ET-1 incubation, resistin protein levels are determined by Western blotting analysis. Levels of resistin protein are increased by about 120%, 130%, and 140% after 10, 50 and 100 nM ET-1 are respectively added.

Together, the present invention provides a medical component for treating insulin resistance related diseases. The component can comprise at least one inhibitor for its decreasing the phosphorylation of downstream signaling molecules stimulated by ET-1, and for its inhibiting resistin mRNA expression stimulated by ET-1. At least one inhibitor of the ET_{A}R and of downstream signaling molecules, such as ERK1/2, JNKs, AKT, and STATS proteins, can be selected.

According to one embodiment of the present invention, it provides a medical component for treating insulin resistance related diseases. The medical component may be applied to treat insulin resistance-related diseases, such as hypertension, inflammation, metabolic syndrome, dislipidemia, glucose intolerance, type II diabetes, hyperuricemia, central obesity, blood coagulation system defect, blood hypercoagulation, hyperandrogenism, fatty liver, or coronary heart disease, but not limited thereto. Furthermore, the medical component can comprise at least one of the pharmaceutically acceptable carriers, diluents, vehicle substrates, and adjuvants for the application use by the way of oral, intramuscular, or intravenous administration, but not limited thereto.

As described above, the present invention provides a medical composition for treating insulin resistance related diseases. This composition comprises at least one inhibitor for decreasing the activity of ET_{A}R and the phosphorylation of downstream signaling molecules stimulated by ET-1. This composition can suppress the ET-1-stimulated resistin mRNA expression through inhibiting the ET-1-stimulated phosphorylation of downstream signaling molecules. Thus, it achieves the purpose of treating insulin resistance-related diseases.

The present invention has been described with a preferred embodiment thereof and it is understood that many changes and modifications to the described embodiment can be carried out without departing from the scope and the spirit of the invention that is intended to be limited only by the appended claims.

What is claimed is:

1. A pharmaceutical composition for treating disorders associated with insulin resistance, comprising:
at least one inhibitor for decreasing the phosphorylation of downstream signaling molecules stimulated by ET-1, in order to be an effective component to inhibit the gene expression level of resistin, wherein the downstream signaling molecules comprise ERK1/2, JNKs, AKT or STAT3, and wherein the inhibitor is selected from at least one antagonist of the downstream signaling molecule.

2. The pharmaceutical composition for treating disorders associated with insulin resistance according to claim 1, wherein the inhibitor is selected from an inhibitor of ET_{A}R.

3. The pharmaceutical composition for treating disorders associated with insulin resistance according to claim 1, wherein the antagonist of the ERK1/2 is selected from 1,4-diamino-2,3-dicyano-1,4-bis (o-aminophenylmercapto) butadiene (U0126) or 2-(2-amino-3-methoxyphenyl)-4H-1-benzopyran-4-one (PD98059).

4. The pharmaceutical composition for treating disorders associated with insulin resistance according to claim 1, wherein the antagonist of the JNKs is selected from anthrapyrazolone (SP600125).

5. The pharmaceutical composition for treating disorders associated with insulin resistance according to claim 1, wherein the antagonist of the AKT is selected from 2-(4-morpholinyl)-8-phenyl-4H-1-benzopyran-4-one (LY294002) or wortmannin.

6. The pharmaceutical composition for treating disorders associated with insulin resistance according to claim 1, wherein the antagonist of the STAT3 is tyrphostin (AG490).

7. The pharmaceutical composition for treating disorders associated with insulin resistance according to claim 1, wherein the disorders associated with insulin resistance is selected from hypertension, inflammation, metabolic syndrome, dyslipidemia, glucose intolerance, type II diabetes, hyperuricemia, central obesity, blood coagulation system defects, high blood coagulation, hyperandrogenism, fatty liver, or coronary heart disease.

8. The pharmaceutical composition for treating disorders associated with insulin resistance according to claim 1, wherein the pharmaceutical composition further comprises at least one of pharmaceutically acceptable carriers, diluents, carrier substances and adjuvants.

9. The pharmaceutical composition for treating disorders associated with insulin resistance according to claim 1, wherein the pharmaceutical composition is a formulation of oral type, intramuscular injection type or intravenous injection type.
